(19) United States
(12) Patent Application Publication
(10) Pub. No.: US 2021/0379580 A1
(43) Pub. Date: Dec. 9, 2021

(54) EASILY DEPLOYED LATERAL FLOW TEST INSTRUMENT AND MANAGEMENT SYSTEM FOR ANTIGEN DETECTION IN HIGH THROUGHPUT TEST CENTERS

(71) Applicants: Sameh Sarhan, Santa Clara, CA (US); Mohammad Iman Sadreddin, Union City, CA (US)

(72) Inventors: Sameh Sarhan, Santa Clara, CA (US); Mohammad Iman Sadreddin, Union City, CA (US)

(73) Assignee: Xtrava Inc, Santa Clara, CA (US)

(21) Appl. No.: 17/340,067

(22) Filed: Jun. 6, 2021

Related U.S. Application Data
(60) Provisional application No. 63/035,844, filed on Jun. 8, 2020.

(51) Int. Cl. B01L 3/00; G01N 33/543

(52) U.S. Cl. CPC B01L 3/5023; B01L 2300/0825; B01L 2200/027; G01N 33/54386

(57) ABSTRACT

A low-cost immunoassay apparatus and system that facilitates the operation of efficient high-volume microbial disease testing centers is described. Included is a highly accurate and sensitive lateral flow assay instrument, complete with internal environmentally controlled chamber, a through-sample spectrographic reader, and cloud-connected flexible pre- and post-test calibration and data analysis. Also included is a highly integrated cloud-connected smartphone application that manages the flow and procedure of each patient before and during his visit to a testing center.

### CROSS-REFERENCE TO RELATED APPLICATION

Priority is claimed under 37 CFR 1.78 and 35 USC 119(e) to U.S. Provisional Application 63/035,844 (XT2006071), filed 8 Jun. 2020, which is incorporated by reference.

### TECHNICAL FIELD

This disclosure relates generally to instruments that determine the presence and/or quantity of chemical compounds. More specifically, this disclosure pertains to a highly accurate and efficient but low-cost lateral flow instrument, automatic electronic reader and data management system. The principal intended use is to test persons of interest and others who may be infected by viruses or other dangerous foreign microbes in very high-volume test centers.

### BACKGROUND

The short-term increase COVID-19 testing throughput is a matter of public health and safely reopening the economy. A successful implementation will require a multi-disciplinary approach. The instant specification on discloses a solution that can effectively enable the fast deployment of large-scale walk-in and drive-thru testing that supports COVID-19 testing and that for other microbial threats to health.

### BRIEF SUMMARY

This Brief Summary is provided as a general introduction to the Disclosure provided by the Detailed Description and Figures, summarizing some aspects of the disclosed invention. It is not a detailed overview of the instant disclosure and should not be interpreted as necessarily identifying key elements of the invention, or otherwise characterizing the scope of the invention disclosed in this Patent Document.

This disclosure includes a digital lateral flow test instrument that combines a COVID-19 antigen based fluorescent test strip with a low-cost compact and portable connected analyzer. It also includes non-fluorescent test strips as needed for COVID 19 virus, influenza virus, and any other microbial heath threats. It is accompanied by a cloud-connected smartphone application that guides at-risk users through registration, finding a test site, making and negotiating an appointment and receiving test results. The smartphone application has the capability of using machine vision to guide and provide real-time validation of a self-administered test at a test center or at-home.

Intended Use: The example embodiment system herein described includes a holistic COVID-19 digital testing platform and patient management system that addresses and mitigates many of the bottlenecks which can hinder the effective deployment of large-scale walk-in and drive-thru testing. For example, the platform facilitates quickly turning any parking lot, stadium, or open space into a large-scale makeshift testing facility.

### BRIEF DESCRIPTION OF DRAWINGS

For a more complete understanding of this disclosure and its features, reference is now made to the following description, taken in conjunction with the accompanying figures, in which:

FIG. 1 shows the key specifications of the tests to be performed.

FIG. 2 shows data illustrating the effectiveness of Lanthanide particle technology for detection of the Malaria Antigen.

FIG. 3 shows comparison Chlamydia detection data for various nanoparticles.

FIG. 4 shows key specifications of the Lateral Flow instrument reader described herein.

FIG. 5 compares the results of a typical lateral flow benchtop reader with the (Xtrava Health) reader described herein.

FIG. 6 shows accurate detection even at low dilution of the Lateral Flow reader described herein.

FIG. 7 shows the operations flow chart of the high throughput testing center described herein.

### DETAILED DESCRIPTION

The various figures, and any various embodiments used to describe the principles of the present invention in this patent document are by way of illustration only and should not be construed in any way to limit the scope of the invention. Those skilled in the art will understand that the principles of the invention may be implemented in any type of suitably arranged device or system.

The digital lateral flow test instrument platform consists of 3 key components A) Fluorescent based antigen test, B) Low-cost portable and reusable digital test reader, C) cloud-connected mobile application for the patient. These components address the following important current impediments to high throughput testing:

Fluorescent-based antigen test:
Availability of low-cost tests
Improves sensitivity, limit of detection, reliability, accuracy, speed and throughput as compared with standard lateral flow assay tests

Low-cost portable and reusable digital test reader:
Availability of easy to deploy testing equipment
Improves limit of detection, reliability, accuracy, speed and throughput
Enables mobile integration that allows for better patient experience, high throughput automated authentication and test results reporting

Cloud-connected mobile application for use by the patient:
Allows healthcare professionals to focus on sample collection and test administration.
Reduces test time by allowing users to register beforehand to minimize queue times
Eliminates much manual administrative work and simplifies sample collection, testing and automating result reporting

The cloud-connected mobile application for use by the patient also
Finds a test site
Makes appointment for the test
Ushers the patient at the test site
Assists with the test
Informs the patient and health professional that a valid test is complete; so the patient knows when to leave the site.

Also, the mobile application has
the ability to integrate with existing telehealth systems
Smartphone application uses machine vision to guide and provide real-time validation of a self-administered test with minimal or no healthcare professional supervision.
User authentication for saving and sharing of test results
Facilitates the management of large-scale test sites.
Includes tracking of test results, inventory and scheduling

Platform Component “A” digital lateral flow test instrument fluorescent-based antigen test also uses the methods of Quidel's Sophia 1 and Sophia 2 tests, which use Lanthanide Particles instead of visual particles for detection. The advantages are:
addresses problems of sensitivity, stability and manufacturing issues affecting current chemical labels;
easy to use and cost-effective;
high sensitivity, enhanced detection (50 to 1000-fold detection level increase over visual particle assay);
stable fluorophores with Stokes shift of over 180 nano-meters;
no monochromatic filter required to read results;
lanthanide particle technology can readily be substituted into current particle-labeled products;
four (4) lanthanides emit different wavelengths (different colors) providing a unique multiplexing option;
novel lanthanide particle allows covalent coupling of binding partner to the particle;
lanthanide is not bound to binding partner;
chemistry without chelation allows for more stable, robust chemical labels; and
result can be quantified for use in a quantitative or qualitative test format.

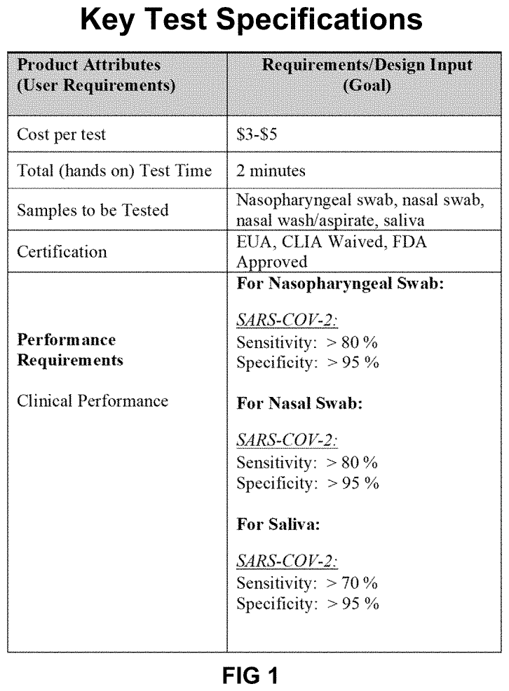

FIG. 1 shows the key specifications of the tests to be performed. The cost per test is only $3.00 to $5.00, and the total time the patient is occupying personnel taking the test is 2 minutes.

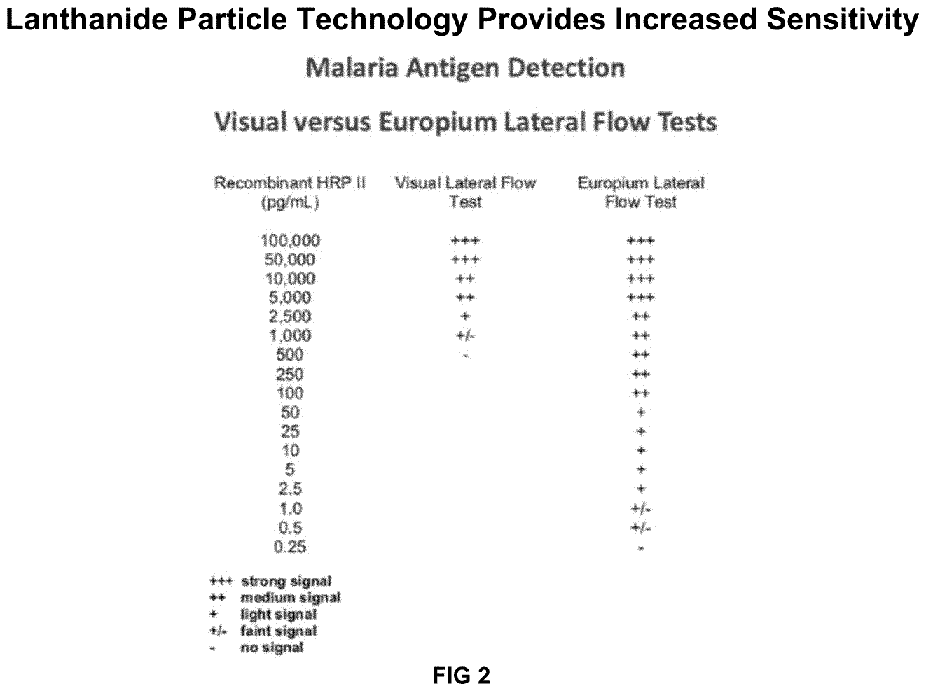

FIG. 2 shows data illustrating the effectiveness of Lanthanide particle technology for detection of the Malaria Antigen. The Recombinant HRP II test has been shown to be 2000 times sensitive than visual reading of the strip.

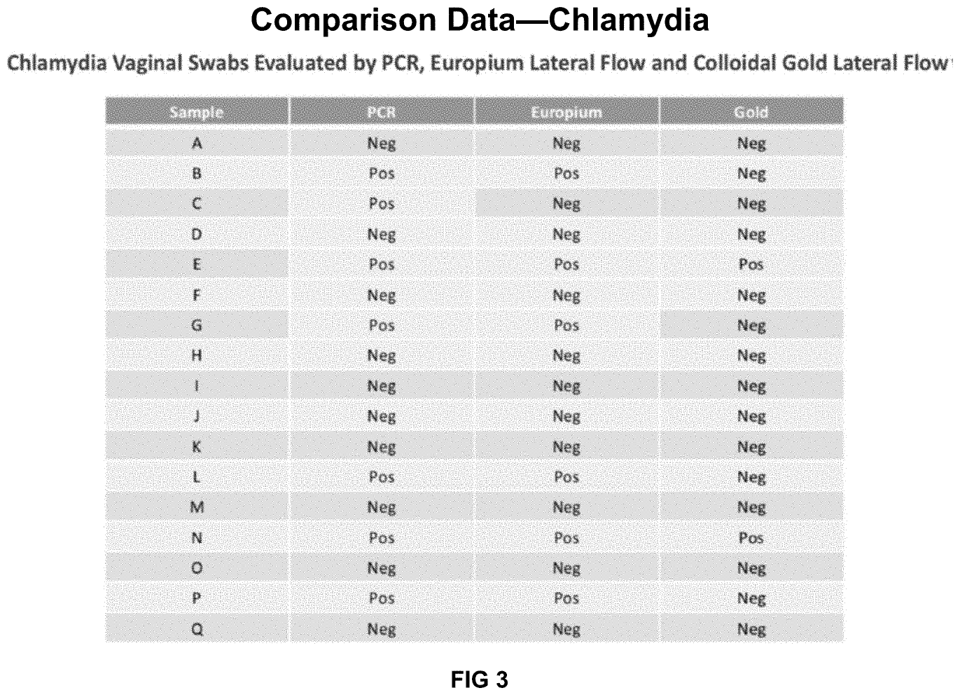

FIG. 3 shows comparison Chlamydia detection data for various nanoparticles.

Platform Component B, digital and connected reusable Lateral Flow Test Reader is disclosed in two patent applications having the same assignee as the instant application. They are “Methods to Register and Interpret Lateral Flow Test Strip Assay Measurements by Transmission of Multi-Wavelength Visible and Near-Visible Light”, Ser. No. 17/107,903; and “Method and Apparatus to Provide Connected, In Situ, Comprehensive, and Accurate Lateral Flow Assays”, Ser. No. 17/323,780. Both applications are hereby incorporated by reference.

The lateral flow assay system described in the above patent applications includes a high-sensitivity solid state spectral sensing solution that enables a cost-effective and portable lateral flow test reader supporting both visible and fluorescent measurements. It provides comparable performance to benchtop/professional readers; although it is much smaller and cheaper to build. It is ultra-compact in size and battery powered.

The apparatus provides fast results (5-10 minutes), including time for sample preparation and collection. Even with the features that provide benchtop accuracy, the high throughput testing characteristic of the portable lateral flow method is retained.

The lateral flow test strip cassette inserted into the test instrument apparatus is safely removed and disposed of following each test.

The environmental chamber is also removed from the instrument for washing and sanitizing and later re-use. The entire instrument is ready for the next test in a matter of seconds.

Mobile Integration using NFC/BLE along with the user's smartphone application eliminate the need for any data entry by a healthcare professional. Automatic synchronizing is performed when the reader is in the presence of the user's smartphone application or a connected Hub station.

Test results are automatically sent to the patient's smartphone application via cloud integration. Decentralized testing allows millions of tests to be run simultaneously.

Artificial Intelligence operations “edge-computing” performed within the reader equipment enables:
Reading, processing, and securely sharing test results
Detection and notification of user concerning errors administering the test
Rejects unauthorized and non-authenticated tests

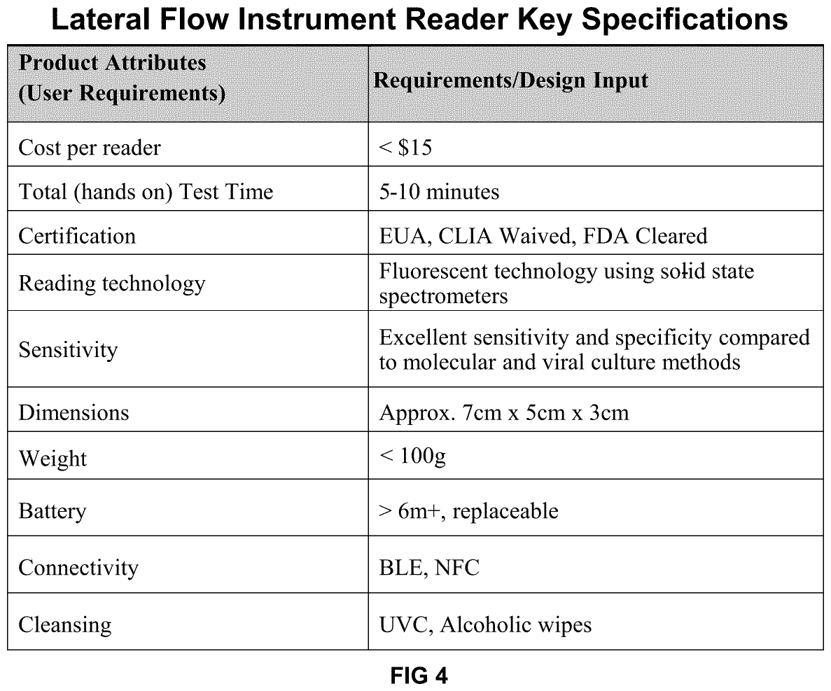

FIG. 4 shows additional key specification of the lateral flow assay apparatus as integrated with the reader.

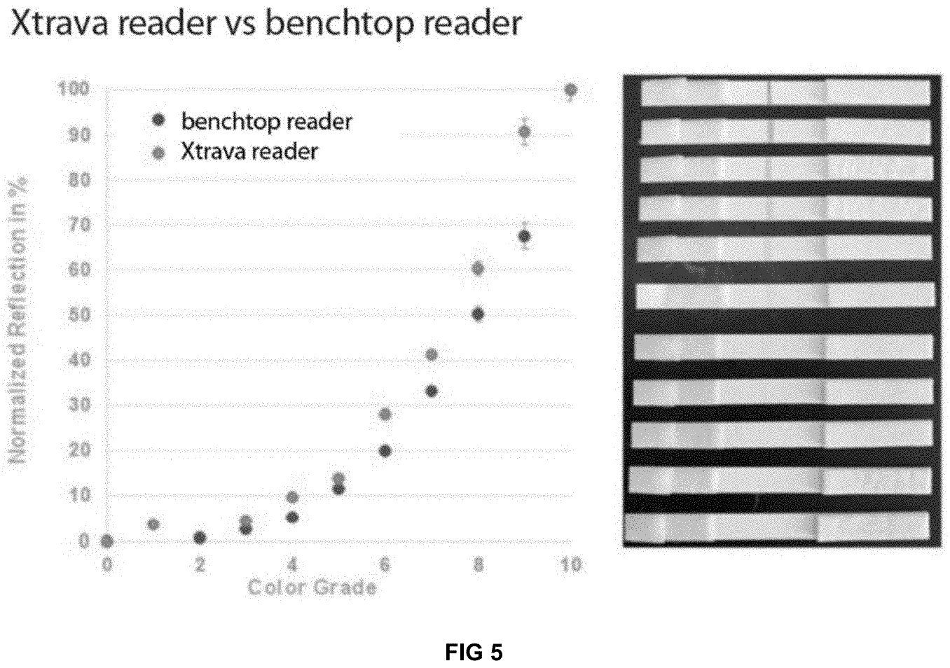

FIG. 5 compares the results of a typical lateral flow benchtop reader with the (Xtrava Health) reader described herein.

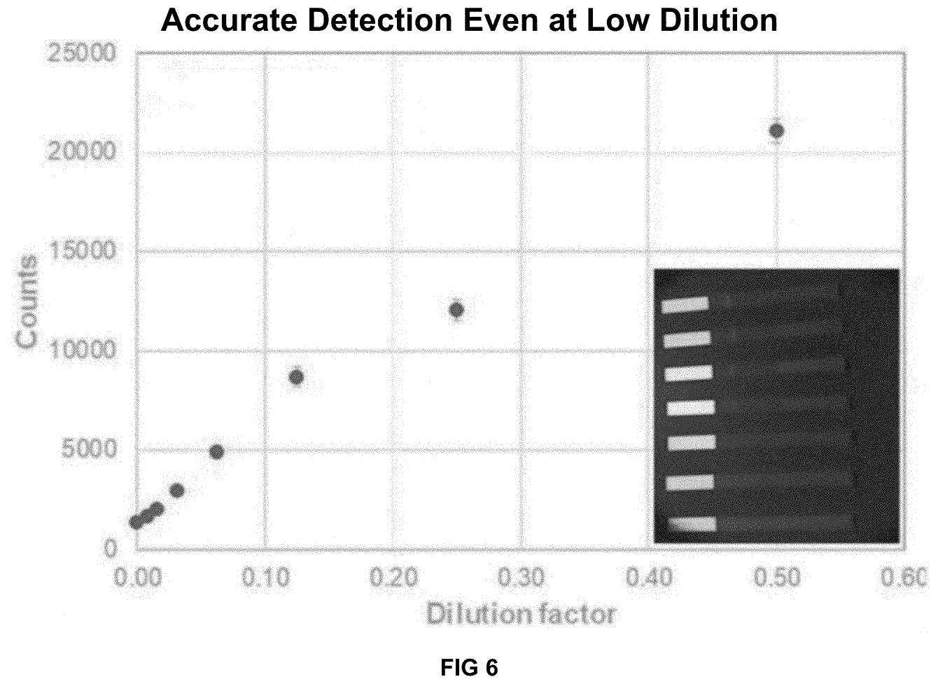

FIG. 6 shows accurate detection even at low dilution of the Lateral Flow reader described herein.

Platform Component C: Smart Phone Application that uses the internet “Cloud”

This application for smartphones and tablet computers provides user registration and authentication. It securely shares user test results; no personally identifiable information is ever uploaded to the cloud.

This application automatically locates test sites and supports making appointments for test.

During the test, patient's smartphone application temporarily pairs with the assay test apparatus via NFC/BLE. It also communicates with the health professional's test and reporting station. It assists with test site administration, including but not limited to inventory management.

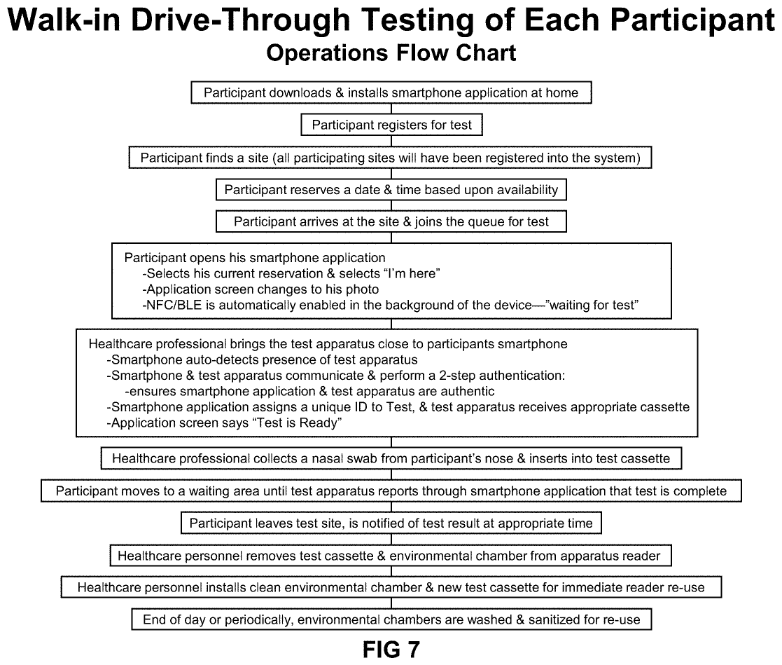

FIG. 7 shows the operations flow chart of the high throughput testing center described herein.

Test Site Scenario
Large-scale walk-in/drive-thru testing of 1000 people.
Sample collection and test administration is performed by a healthcare professional.
Number of healthcare professionals: 20
Average sample collection time: 2 minutes per participant
Switching to next participant: 1 minute
Site throughput: 100 participants per hour (assuming 20% lost time/overhead)

The details provided in the above description describe particular implementations of the systems for performing the measurements described. Other embodiments could be implemented in any other suitable manner. For example, particular voltages, frequencies, noise levels, gains, resistances, capacitances, and other values may be described. These values are for illustration only. It may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An integrated immunoassay instrument and management system comprised of
a lateral flow assay instrument,
a solid state spectral-sensing lateral flow test strip reader;
a smartphone application that guides the patient through all steps to be tested by the immunoassay instrument;
where the integrated immunoassay instrument and management system facilitates operation of efficient high-volume microbial disease testing centers.
